(19) United States
(12) Patent Application Publication
(10) Pub. No.: US 2023/0284878 A1
(43) Pub. Date: Sep. 14, 2023

(54) WHITE BALANCE APPARATUS

(71) Applicant: 270 SURGICAL LTD., Netanya (IL)

(72) Inventors: Golan SALMAN, Atlit (IL); Hadar SCHWARCZ, Netanya (IL); Ruvi FINEBERG, Hod Ha'sharon (IL)

(21) Appl. No.: 18/015,386

(22) PCT Filed: Jun. 28, 2021
(86) PCT No.: PCT/IL2021/050786

§ 371 Date: Jan. 10, 2023

Related U.S. Application Data
(60) Provisional application No. 63/050,849, filed on Jul. 12, 2020.

(51) Int. Cl. A61B 1/00; A61B 1/05; H04N 23/88

(52) U.S. Cl. CPC A61B 1/00096; A61B 1/05; A61B 1/00057; A61B 1/000095; A61B 1/00177; A61B 1/00181; A61B 1/00045; H04N 23/88; A61B 2090/0811

(57) ABSTRACT

Provided is an apparatus for performing simultaneous white balance of a plurality of cameras, located at a distal tip of an endoscope. Further provided are systems including the white balance apparatus and methods of using the same.

### TECHNICAL FIELD

The present disclosure relates generally to a white balance apparatus for an endoscope having a plurality of cameras. Further provided are methods of manufacturing the apparatus and methods of using the same.

### BACKGROUND

An endoscope is a medical device used to image an anatomical site (e.g. an anatomical/body cavity, a hollow organ). Unlike some other medical imaging devices, the endoscope is inserted into the anatomical site (e.g. through small incisions made on the skin of the patient). An endoscope can be employed not only to inspect an anatomical site and organs therein (and diagnose a medical condition in the anatomical site) but also as a visual aid in surgical procedures. Medical procedures involving endoscopy include laparoscopy, arthroscopy, cystoscopy, ureterostomy, and hysterectomy.

White balance (WB) is the process removing unrealistic color casts created as an artifact by digital imaging units (cameras), so that objects which appear white under different light sources or conditions are rendered white in the captured image/video. The white balance process considers inter alia, the color temperature of the light source and the white balance therefore improves the quality of the acquired image/video under a wide range of lighting conditions and light sources.

White balance process is particularly important for endoscopic procedures, where the images and/or video streams obtained from internal organs need to be as clear as possible, and hence color adjustment is critical for proper visualization.

There is thus a need in the art for apparatuses and methods for performing an accurate white balance for cameras of an endoscope, in particular, for endoscope system having more than one camera, wherein each of the cameras is placed in different orientation in the distal tip of the endoscope.

### SUMMARY

Aspects of the disclosure, according to some embodiments thereof, relate to an advantageous white balance apparatus, for use with an endoscope having a plurality of cameras at the endoscope distal tip. The disclosed white balance apparatus, allows an efficient and accurate white balance procedure to the plurality of cameras of the endoscope, in an accurate and simultaneous fashion. The disclosed white balance apparatus is advantageous as it is accurate, cost efficient, customizable and easy to use. The disclosed white balance apparatus provides means to allow simultaneous white balance calibration to the plurality of cameras of the endoscope, thereby ensuring optimal images and videos to be obtained during surgical operation. The various apparatuses disclosed herein may be disposable or reusable and may advantageously be sterilizable.

According to some embodiments, there is provided a white balance apparatus for a distal tip of an endoscope which includes at least two cameras, the white balance apparatus includes a shell which is at least partially enclosing an internal space, the shell includes an opening configured to allow the insertion of the distal tip into the internal space, such that each of the cameras faces a portion of the internal wall of the shell at a predefined distance therefrom, to thereby allow simultaneous white balancing of the at least two cameras.

According to some embodiments, there is provided a system for white balance of at least two cameras located at a tip of an endoscope, the system includes a white balance apparatus having a shell at least partially enclosing an internal space, the shell includes an opening configured to allow the insertion of the endoscope tip into the internal space, such that each of said cameras faces a portion of the internal wall of the shell at a predefined distance therefrom; and a main control unit configured to receive image data from each of the cameras while being placed/located within the apparatus, and execute a white balance processing on the images.

According to some embodiments, there is provided a method for performing white balance for images obtained simultaneously from least two cameras located at an endoscope tip, the method includes: inserting the endoscope tip into a white balance apparatus having a shell at least partially enclosing an internal space, the shell includes an opening configured to allow the insertion of the endoscope tip into said internal space, such that each of the cameras faces a portion of the internal wall of the shell at a predefined distance therefrom; verifying the positioning of the endoscope tip within the inner space of the apparatus, such that each of the cameras is placed at a predetermined distance from the internal wall of the shell of the apparatus; obtaining one or more images from the cameras, and processing the one or more images to perform white balance using a processor on a main control unit.

According to some embodiments, there is provided a white balance apparatus for a distal tip of an endoscope, said distal tip includes at least two cameras, the white balance apparatus includes an external shell at least partially enclosing an internal space, the external shell includes an opening configured to allow the insertion of the distal tip into said internal space, such that each of said at least two cameras faces a portion of an internal shell surface of the internal space at a predefined distance therefrom, to thereby allow simultaneous white balancing of said at least two cameras.

According to some embodiments, the opening further includes an indicator (apparatus indicator) configured to indicate the extent of insertion of the distal tip into the internal space of the apparatus.

According to some embodiments, the indicator may include a marking, a rim, a groove and/or a sensor.

According to some embodiments, the indicator may be configured to facilitate the positioning of the distal tip within the internal space, such that each of said at least two cameras is placed at the predetermined distance from the internal shell surface of the apparatus.

According to some embodiments, external (ambient) light is essentially prevented from entering the internal space when the distal tip is positioned with the internal space.

According to some embodiments, the internal space may include a cylindrical, round, or spherical shape.

According to some embodiments, the apparatus is substantially spherical and comprises a semi-flexible structure.

According to some embodiments, the apparatus may be made of thermoplastic elastomer (TPE) and/or a thermoset elastomer.

According to some embodiments, the apparatus may be made of silicone. According to some embodiments, the silicone may include 40 shore silicon.

According to some embodiments, the apparatus may be constructed by press molding, injection molding and/or machining.

According to some embodiments, the apparatus may be substantially cylindrical or tube-like and comprises at least partially rigid walls. According to some embodiments, the apparatus may further include an outer shell made of or includes glass, and/or transparent plastic, and an external coating. According to some embodiments, the external coating is white. According to some embodiments, the external coating includes Polytetrafluoroethylene (PTFE). According to some embodiments, thickness of the shell may be determined based on the light focus distance.

According to some embodiments, the predetermined distance between each of the at least two cameras and the internal shell surface of the apparatus is a working distance of each of the at least two cameras.

According to some embodiments, a distance between an outer window of each of the at least two cameras and the internal shell surface of the apparatus may be in the range of about 1-150 mm.

According to some embodiments, the distance between the outer window of each of the at least two cameras and the internal shell surface of the apparatus may be in the range of about 30-60 mm.

According to some embodiments, each of the at least two cameras may be associated with at least one illumination component.

According to some embodiments, the at least two cameras include a front camera and a first side-camera.

According to some embodiments, the at least two cameras further include a second side-camera, wherein the first side-camera and the second side-camera are positioned on opposite sides of the endoscope tip, and wherein the first side-camera is positioned distally relative to the second side-camera.

According to some embodiments, the at least two cameras may provide at least about 270 degrees horizontal field-of-view (FOV) of a target area within an anatomical cavity into which the endoscope is inserted, after a white balance calibration is performed utilizing the white balance apparatus.

According to some embodiments, the at least one illumination component may be or may include a discrete light source.

According to some embodiments, each of the at least two cameras may include a sensor configured to be associated with a main control unit having a suitable white balance circuit for executing a white balance processing of images obtained by the at least two cameras, while the distal tip of the endoscope is placed within the white balance apparatus.

According to some embodiments, there is provided a system for white balance of at least two cameras located at a distal tip of an endoscope, the system includes: a white balance apparatus including an external shell at least partially enclosing an internal space, the external shell including an opening configured to allow the insertion of the distal tip into said internal space, such that each of said at least two cameras faces a portion of an internal shell surface of the internal space at a predefined distance therefrom; and a main control unit configured to receive image data from each of the at least two cameras while placed within the apparatus, and execute a white balance processing on said images.

According to some embodiments, the system may further include a display configured to display one or more images and/or parameters related to the white balance processing.

According to some embodiments, there is provided a method for performing white balance for images obtained simultaneously from at least two cameras located at a distal tip of an endoscope, the method includes one or more of the steps of:
inserting the distal tip of the endoscope into a white balance apparatus having an external shell at least partially enclosing an internal space, the external shell comprising an opening configured to allow the insertion of the distal tip into said internal space, such that each of said at least two cameras faces a portion of an internal shell surface of the internal space at a predefined distance therefrom,
verifying the positioning of the distal tip within the internal space of the apparatus, such that each of the at least two cameras is placed at a predetermined distance from the internal shell surface of the apparatus;
obtaining one or more images from the at least two cameras, and
processing the one or more images to perform white balance using a processor on a main control unit.

According to some embodiments, the method may further include storing in memory white balance parameters.

According to some embodiments, the white balance may be performed for a predetermined period of time. According to some embodiments, the period of time may be in the length of about 1-10 seconds.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1—schematically depict a rigid endoscope, according to some embodiments;

FIG. 2—schematically depicts a medical imaging system including an endoscope having a plurality of cameras, according to some embodiments;

FIG. 3—schematically depicts an elongated shaft of an endoscope, and a field-of-view provided by cameras positioned in a distal section (tip) of the elongated shaft, which is a specific embodiment of the endoscope of FIG. 1;

FIG. 4—shows a perspective view of a front and side cameras disposed at a distal tip of an endoscope, according to FIG. 3;

FIGS. 5A-D show different views of an apparatus for white balance of a distal tip of an endoscope, according to some embodiments. FIG. 5A show a perspective view of a white balance apparatus; FIG. 5B shows a side view of white balance apparatus; FIG. 5C shows a bottom view of white balance apparatus; FIG. 5D shows a perspective view of a cross section of a white balance apparatus.

FIG. 6A—illustrates a cross section of a perspective view of a white balance apparatus of FIGS. 5A-5D having a distal tip of an endoscope which includes a plurality of cameras inserted thereto and each camera provided a field-of-view as shown in FIG. 3, according to some embodiments;

FIG. 6B illustrates a cross section of a side view of a white balance apparatus of FIGS. 5A-5D having a distal tip of an endoscope which includes a plurality of cameras inserted thereto and each camera provided a field-of-view as shown in FIG. 3, according to some embodiments;

FIG. 6C illustrates an exemplary perspective view of a field of view of a side camera of a distal tip of endoscope of FIG. 3 which includes a plurality of cameras, inserted into a white balance apparatus of FIGS. 5A-5D, according to some embodiments;

FIG. 7A illustrates a white balance apparatus, according to some embodiments;

FIG. 7B illustrates a white balance apparatus, according to some embodiments; and

FIG. 8 illustrates steps in a method for performing white balance of images obtained from a plurality of cameras of a distal tip of an endoscope, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

According to some embodiments, there is provided herein an advantageous white balance apparatus for an endoscope having two or more cameras at a distal end (tip) thereof. The advantageous white balance apparatus allows an efficient and accurate white balance processing, preferably simultaneously, to all cameras of the endoscope, albeit differences between the cameras, such as, that each camera faces a different direction of view and having a different field of view.

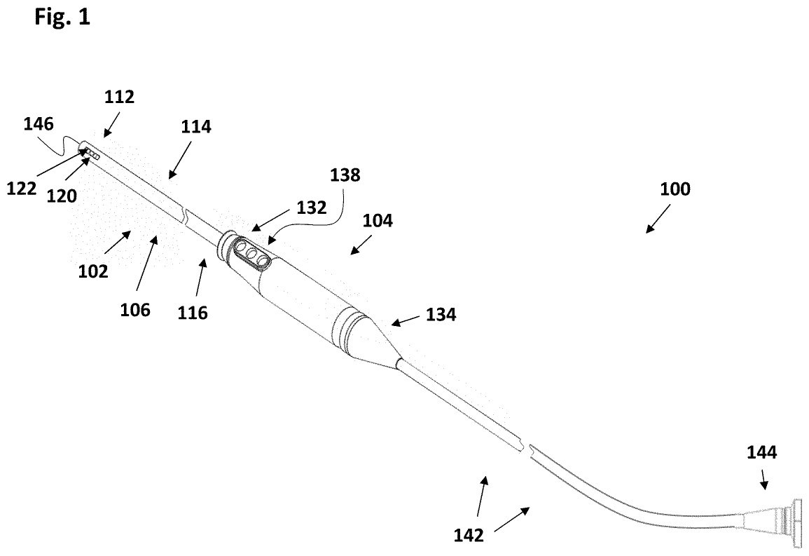

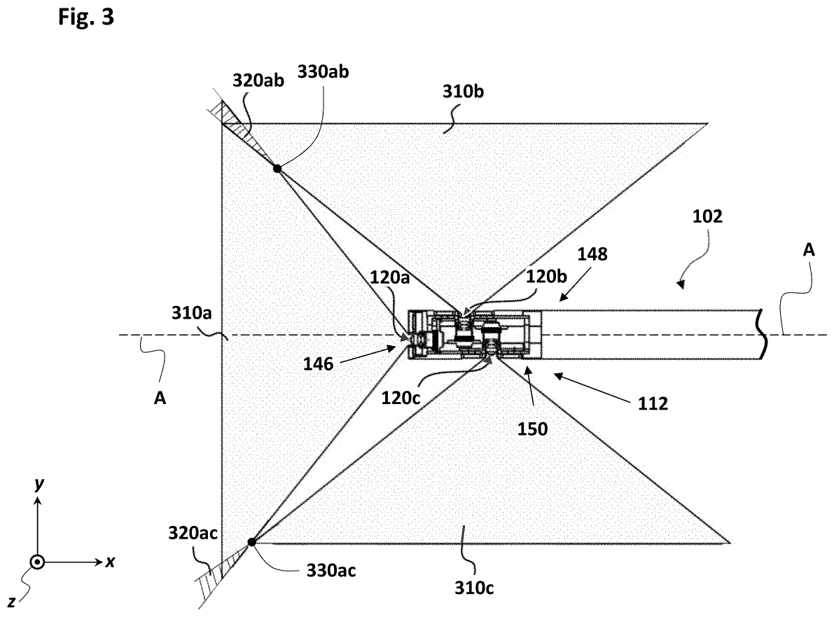

Reference is now made to FIG. 1, which schematically depict a rigid endoscope, according to some embodiments. As shown in FIG. 1, endoscope 100 includes an elongated shaft 102, configured to be inserted into an anatomical site (e.g. an anatomical cavity), and a handle 104, configured to be held by a user (e.g. a surgeon or a robotic arm) of endoscope 100 and to facilitate guiding and manipulation of elongated shaft 102 (particularly a distal section thereof) within the anatomical site. Shaft 102 includes a shaft body 106, e.g. a rigid tubular member. Shaft 102 includes a shaft distal section 112, a shaft central section 114, and a shaft proximal section 116 (i.e. a distal section, a central section, and a proximal section, respectively, of shaft 102). Shaft distal section 112 includes at least two cameras 120 (e.g. a front camera, as seen for example in FIG. 3, and at least one side camera) and illumination components 122, such as light emitting diodes (LEDs). According to some embodiments, each of illumination components 122 is or includes a discrete light source. According to some embodiments, wherein illumination components 122 include LEDs, the LEDs may include, for example, one or more white light LEDs, infrared LEDs, near infrared LEDs, an ultraviolet LED, and/or a combination thereof. It is noted that in embodiments wherein illumination components include LEDs configured to produce light outside the visible spectrum (e.g. an infrared spectrum, a UV spectrum), cameras 120 may include suitable sensors configured to detect such type of light (e.g. infrared light, ultraviolet). That is, cameras 120 will have capacities of e.g. infrared cameras and so on. According to some embodiments, the illumination components may include the distal tips of respective optical fibers (not shown).

The handle 104 may include a user control interface 138 configured to allow a user to control endoscope 100 functions. User control interface 138 may be functionally associated with cameras 120 and illumination components 122 via an electronic coupling between shaft 102 and handle 104. According to some embodiments, user control interface 138 may allow, for example, to control zoom, focus, multifocal views, record/stop recording, freeze frame functions, etc., of cameras 120 and/or to adjust the light intensity provided by illumination components 122.

Each of cameras 120 may include a sensor, such as a charge coupled device (CCD) image sensor or a complementary metal oxide semiconductor (CMOS) image sensor, and a camera lens (e.g. an extreme wide-angle lens) or a lens assembly. Cameras 120 may be configured to provide a continuous/panoramic/surround field-of-view (FOV), as elaborated on below in the description of FIG. 3.

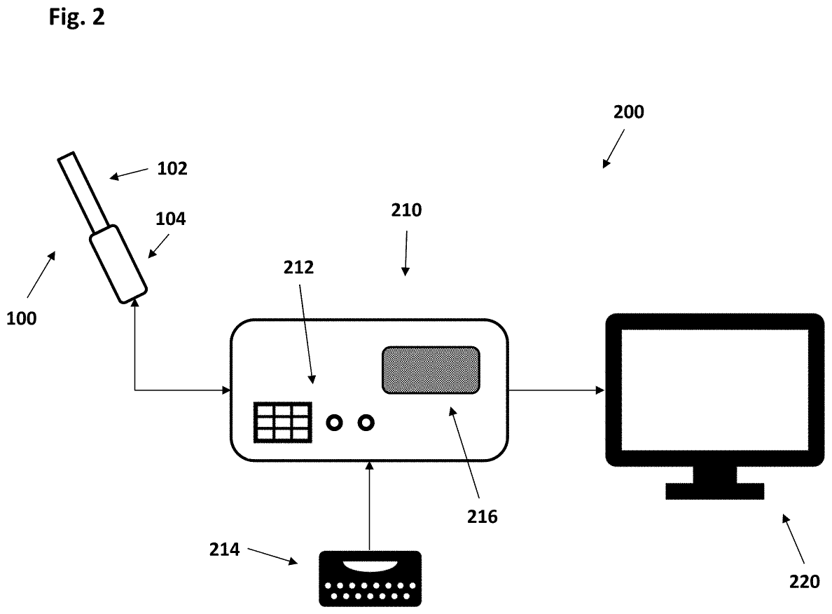

Reference is now made to FIG. 2, which schematically depicts a medical imaging system 200, according to some embodiments. Medical imaging system 200 includes endoscope 100, a main control unit 210, and a monitor 220. According to the convention adopted herein, a same reference numeral in FIGS. 1, 2 and 3, refers to the same object (e.g. device, element).

As shown in FIG. 2, Endoscope 100 and monitor 220 may each be functionally associated with main control unit 210. Main control unit 210 includes processing circuitry (e.g. one or more processors and memory components) configured to process data from at least two cameras 120 (not shown in FIG. 2 but depicted in FIG. 1 at a shaft distal section 112), such as to display the captured image, and video streams on monitor 220. In particular, the processing circuitry may be configured to process the data received from each of the at least two cameras 120, such as to produce therefrom video files/streams providing a 3D, panoramic/surround view of the anatomical site, as explained below in the description of FIG. 3. According to some embodiments, the processing circuitry may be configured to process the data received from cameras 120 to produce a combined video stream providing a continuous and consistent (seamless) panoramic view of the anatomical site.

Main control unit 210 may include a user interface 212 (e.g. buttons and/or knobs, a touch panel, a touch screen) configured to allow a user to operate main control unit 210 and/or may allow control thereof using one or more input devices 214, e.g. an external user control interface connectable thereto such as a keyboard, a mouse, a portable computer, and/or even a mobile computational device e.g. a smartphone or a tablet. According to some embodiments, input devices 214 may include a voice controller. According to some embodiments, main control unit 210 may further be configured to partially or even fully operate at least two cameras 120 and illumination components 122 (shown in FIG. 1 at a shaft distal section 112). Some operational aspects may be operated automatically, for example, according to some embodiments, the supply of power to endoscope 100 components, such as at least two cameras 120 and illumination components 122, while other operational aspects or functions may be operated using user interface 212 and/or input devices 214. According to some embodiments, main control unit 210 may include a display 216 (for example, the touch screen and/or another screen) for presenting information regarding the operation of endoscope 100, such as the brightness levels of at least two cameras 120, zoom options, focus, and the like. According to some embodiments, wherein display 216 is a touch screen, display 216 may further allow controlling for example, the zoom, focus, multifocal imaging, selecting images from specific lens assemblies, compiling images from various lens assemblies, creating a multifocal image, record/stop recording functions, freeze frame function, and/or the brightness of at least two cameras 120, and/or to adjust the light intensity of illumination components 122. According to some embodiments, the choice of information presented may be controlled using user interface 212, user control interface 138, and/or input devices 214.

According to some embodiments, endoscope 100 is functionally associated with main control unit 210 via a utility cable 142 (shown in FIG. 1), connected to or configured to be connected to handle proximal section 134, and further configured to be connected to main control unit 210 (via, for example, a plug 144 or a port). Utility cable 142 may include at least one data cable for receiving video signals from at least two cameras 120, and at least one power cable for providing electrical power to at least two cameras 120 and to illumination components 122, as well as to operationally control parameters of at least two cameras 120 and illumination components 122, such as the light intensity. Additionally or alternatively, according to some embodiments, endoscope 100 may include a wireless communication unit (e.g. a Bluetooth antenna or Wi-Fi) configured to communicatively associate endoscope 100 with main control unit 210. According to some embodiments, endoscope 100 is configured to be powered by a replaceable and/or rechargeable battery included therein, i.e. inside handle 104. According to some embodiments, wherein illumination components 122 include distal tips of optical fibers and wherein the light source(s) is positioned in main control unit 210, cable 142 will also include one or more optical fibers configured to guide the light produced by the light source(s) to an optical fiber(s) in handle 104, wherefrom the light will be guided to optical fibers in shaft 102.

Monitor 220 is configured to display images and, in particular, to display multifocal stream videos captured by at least two cameras 120, and may be connected to main control unit 210 by a cable (e.g. a video cable) or wirelessly. According to some embodiments, monitor 220 may be configured to display thereon information regarding the operation of endoscope 100, as specified above. According to some embodiments, monitor 220, or a part thereof, may function as a touch screen. According to some such embodiments, the touch screen may be used to operate main control unit 210. According to some embodiments, images/videos from different cameras (from at least two cameras 120) or from different lens assemblies of the different cameras, may be displayed separately (e.g. side-by-side, picture on picture, in an equal aspect ratio, in an un-equal aspect ratios, in multiple copies of one or more of the video streams, and the like) on monitor 220, and/or may be presented as a single panoramic/surround, optionally multi-focal or 3D image/video. According to some embodiments, user interface 212 and/or input devices 214 and/or user control interface 138 are configured to allow switching between images/videos corresponding to different field of views (of different cameras) and/or of different field of views (obtained from different lens assemblies of one or more sensors). For example, according to some embodiments, wherein at least two cameras 120 include a front camera 120a, a first side camera 120b, and a second side cameras 120c: switching between footage(s) captured by one or more lens assemblies from front camera 120a to footage(s) captured by one or more lens assemblies of first side camera 120b, switching between footage(s) captured by one or more lens assemblies of front camera 120a to footage(s) captured by one or more lens assemblies of second side cameras 120c, or switching between panoramic/surround video(s) generated from the footage(s) of all of cameras 120a, 120b, and 120c to footage captured by one of cameras 120a, 120b, or 120c. Cameras 120a, 120b, and 120c are depicted together in FIG. 3.

According to some embodiments, main control unit 210 may be associated with a plurality of monitors, such as monitor 220, thereby allowing displaying different videos and images on each. For example, main control unit 210 may be associated with four monitors, such as to allow displaying videos from each of cameras 120a, 120b, 120c on three of the monitors, respectively, and a panoramic video (corresponding to the combination of the three videos) on the fourth monitor, which may be wider than the other three. Main control unit may further be used to calibrate one or more of the endoscope cameras, for example, facilitating white balance calibration, utilizing the white balance apparatuses as disclosed herein.

The field-of-view (FOV) provided by endoscope 100 is the combination of the respective FOVs provided by each of at least two cameras 120. At least two Cameras 120 may be configured to provide a continuous and consistent FOV, or at least a continuous and consistent horizontal FOV (HFOV), as explained below.

Reference is now made to FIG. 3, which schematically depicts shaft distal section 112 (of shaft 102) and a combined HFOV provided by front camera 120a, first side-camera 120b, and second side-camera 120c, which is a specific embodiment of the endoscope of FIG. 1. Front camera 120a is positioned within shaft distal section 112 on a front surface 146 of the distal tip (not numbered) of shaft distal section 112, with a lens assembly (not numbered) of front camera 120a being exposed on front surface 146. First side-camera 120b is positioned within shaft distal section 112 on a first side-surface 148 thereof, with a lens assembly (not numbered) of first side-camera 120b being exposed on first side-surface 148. Second side-camera 120c is positioned within shaft distal section 112 on a second side-surface 150 thereof, with a lens assembly (not numbered) of second side-camera 120c being exposed on second side-surface 150. First side-surface 148 is opposite to second side-surface 150. According to some embodiments, first side-camera 120b and second side-camera 120c are not positioned back-to-back. According to some embodiments, the distance between the center-point of first side-camera 120b (i.e. the center of a lens assembly of first side-camera 120b) and front surface 146 is between about 5 millimeters to about 20 millimeters and the distance between the center-point of first side-camera 120b and the center-point of second side-camera 120c may be up to about 10 millimeters.

In some embodiments, the combined HFOV is formed by a front HFOV 310a, a first side-HFOV 310b, and a second side-HFOV 310c of front camera 120a, first side-camera 120b, and second side-camera 120c, respectively. Each of HFOVs 310a, 310b, and 310c lies on the xy-plane. HFOV 310a is positioned between HFOVs 310b and 310c and overlaps with each. A first overlap area 320ab corresponds to an area whereon HFOVs 310a and 310b overlap. In other words, first overlap area 320ab is defined by the intersection of the xy-plane with the overlap region (volume) of the FOVs of front camera 120a and first side-camera 120b. Similarly, a second overlap area 320ac corresponds to an area whereon HFOVs 310a and 310c overlap. A first intersection point 330ab is defined as the point in first overlap area 320ab which is closest to front camera 120a. It is noted that first intersection point 330ab also corresponds to the point in first overlap area 320ab which is closest to first side-camera 120b. Similarly, a second intersection point 330ac is defined as the point in second overlap area 320ac which is closest to front camera 120a. It is noted that second intersection point 330ac also corresponds to the point in second overlap area 320ac which is closest to second side-camera 120c.

According to some embodiments, the combined HFOV spans between about 220 degrees to about 270 degrees, between about 240 degrees to about 300 degrees, or between about 240 degrees to about 340 degrees. Each possibility corresponds to separate embodiments. According to some embodiments, the combined HFOV spans at least about 270 degrees. According to some embodiments, for example, each of HFOVs 310a, 310b, and 310c may measure between about 85 degrees to about 120 degrees, between about 90 degrees to about 110 degrees, or between about 95 degrees to about 120 degrees. Each possibility corresponds to separate embodiments.

According to some embodiments, shaft 102 may measure between about 100 millimeters and about 500 millimeters in length, and shaft body 106 may have a diameter measuring between about 2.5 millimeters and about 15 millimeters. According to some embodiments, front camera 120a may be offset relative to a longitudinal axis A, which centrally extends along the length of shaft 102. According to some embodiments, the distance between second side camera 120c and front surface 146 is greater than the distance between first side camera 120b and front surface 146.

According to some embodiments, front camera 120a may be offset relative to the longitudinal axis A by up to about 0.05 millimeters, up to about 0.1 millimeters, up to about 0.5 millimeters, up to about 1.0 millimeters, up to about 1.5 millimeters, up to about 5.0 millimeters, or up to about 7.0 millimeters. Each possibility corresponds to separate embodiments. According to some embodiments, for example, front camera 120a may be offset relative to the longitudinal axis A by between about 0.05 millimeters to about 0.1 millimeters, about 0.5 millimeters to about 1.5 millimeters, about 1.0 millimeter to about 5.0 millimeters, about 1.5 millimeters to about 5.0 millimeters, or about 1.0 millimeters to about 7.0 millimeters. Each possibility corresponds to separate embodiments. According to some embodiments, first side-camera 120b may be positioned at a distance of up to about 1.0 millimeters, up to about 5.0 millimeters, or up to about 15.0 millimeters from front surface 146. Each possibility corresponds to separate embodiments. According to some embodiments, second side-camera 120c may be positioned at a distance of up to about 1.0 millimeters, up to about 5.0 millimeters, up to about 15.0 millimeters, or up to about 25.0 millimeters from front surface 146, such as to optionally be positioned farther from front surface 146 than first-side-camera 120b. Each possibility corresponds to separate embodiments. According to some embodiments, the positioning of cameras 120 on shaft distal section 112 is selected such as to minimize the space occupied by cameras 120 and reduce the diameter of shaft distal section 112, while affording a continuous and consistent HFOV of at least about 270 degrees.

According to some embodiments, each of cameras 120 is associated with a respective illumination component from illumination components 122, which is configured to illuminate the FOV of the camera. Thus, according to some embodiments, front camera 120a may be associated with a respective front illumination component (not numbered), first side-camera 120b may be associated with a respective first side-illumination component, and second side-camera 120c may be associated with a respective second side-illumination component.

According to some embodiments, not depicted in the figures, cameras 120 include only two cameras, both of which are side cameras with fisheye lenses. In such embodiments, shaft distal section 112 may taper in the distal section, such that the cameras provide a continuous HFOV. According to some embodiments, not depicted in the figures, cameras 120 include only two cameras: a front camera and a side camera.

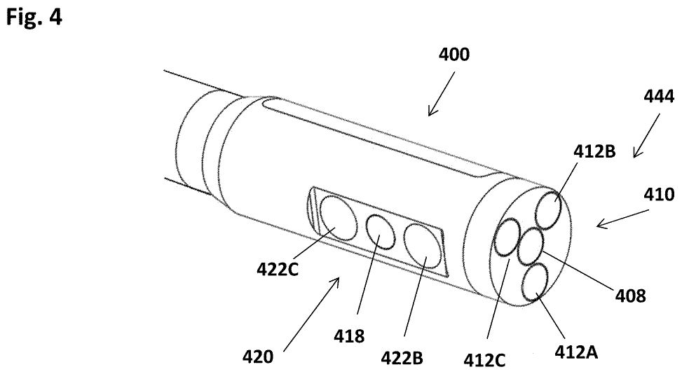

Reference is now made to FIG. 4, which depicts a perspective close up view of a front and side cameras at a tip of an endoscope, according to some embodiments depicted in FIG. 3. As shown in FIG. 4, a distal tip 444 of an endoscope 400, includes a front camera 408, which faces a front view 410 of distal tip 444, and a side camera 418 which faces a side view 420 of distal tip 444. The front and side cameras shown in FIG. 4 are such front camera 120a, first side camera 120b and second side camera 120c which operation is disclosed at FIG. 3. Adjacent to front camera 408 located illumination sources (shown as light sources 412A, 412B and 412C), the illuminations sources may include any type of light, as detailed above. As further shown in FIG. 4, adjacent to side camera 418 are illumination sources 422A-B.

According to some embodiments, each of endoscopes 100, and 400 may be (i) directly maneuvered by a user through the manipulation of handle 104, as well as (ii) indirectly maneuvered, via robotics, e.g. using a robotic arm or other suitable gripping means configured to allow manipulation of handle 104.

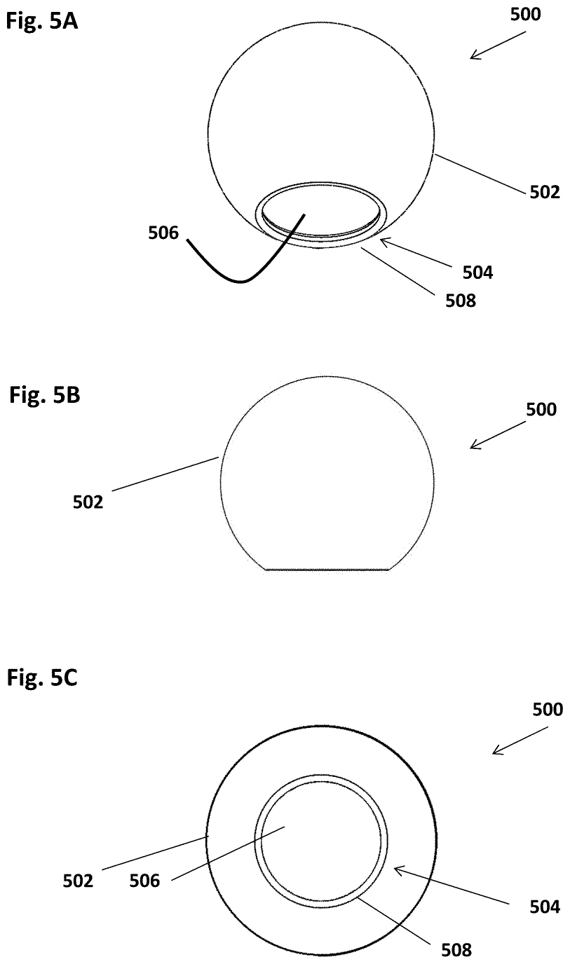

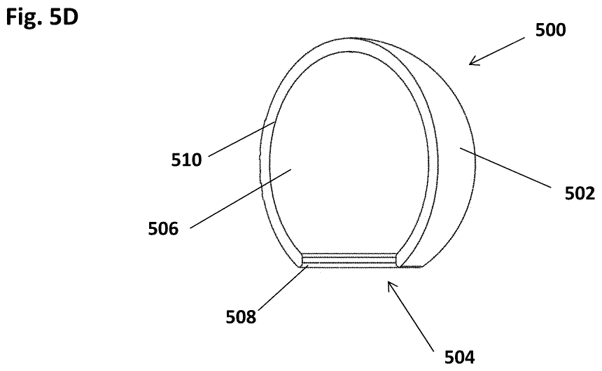

Reference is now made to FIGS. 5A-D which show different views of an apparatus for white balance of a distal tip of an endoscope, according to some embodiments. FIG. 5A show a perspective view of a white balance apparatus 500, having an external shell 502, which at least partially enclose an internal space (volume) 506. The external shell 502 further includes an opening 504, which is configured to allow insertion of a distal tip of an endoscope into the internal space 506 of the white balance apparatus 500. Opening 504 includes on the surface thereof an apparatus indicator (indication) 508, which may be of various types, include, a groove, a rim, a sensor, a visual indication, and the like, to allow identification and/or determination of the position/location/degree of insertion of the distal tip into the internal space. FIG. 5B shows a side view of white balance apparatus 500, showing external shell 502. FIG. 5C shows a bottom view of white balance apparatus 500, showing opening 504, the external shell 502, internal space 506 and apparatus indicator 508. FIG. 5D shows a perspective view of a cross section of white balance apparatus 500, showing the external shell 502, an internal shell surface/face 510, and the internal space 506. Further shown is opening 504 as well as apparatus indicator 508, located on opening 504.

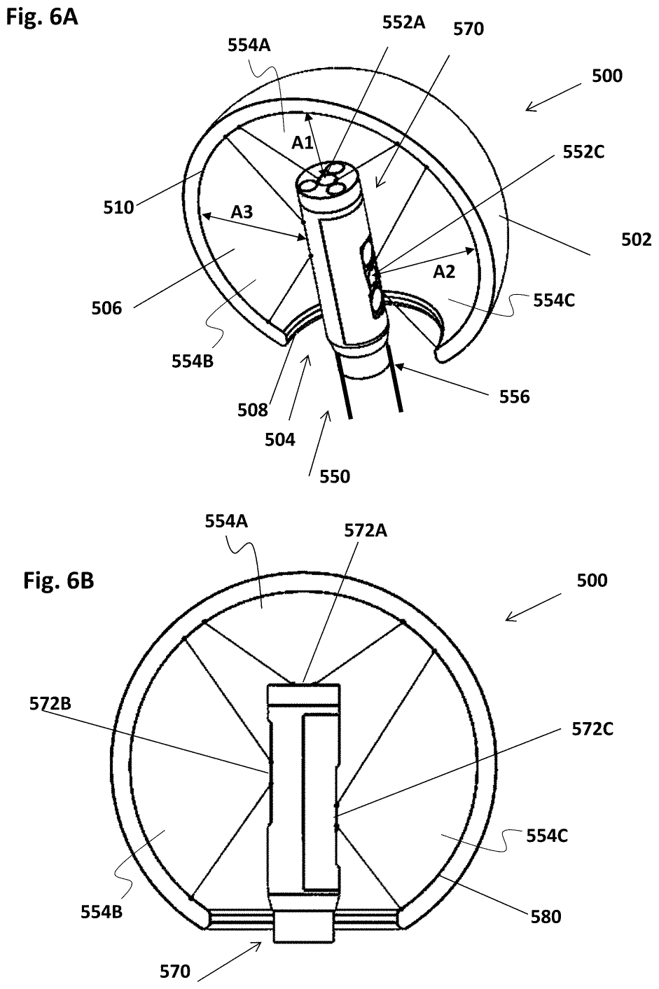

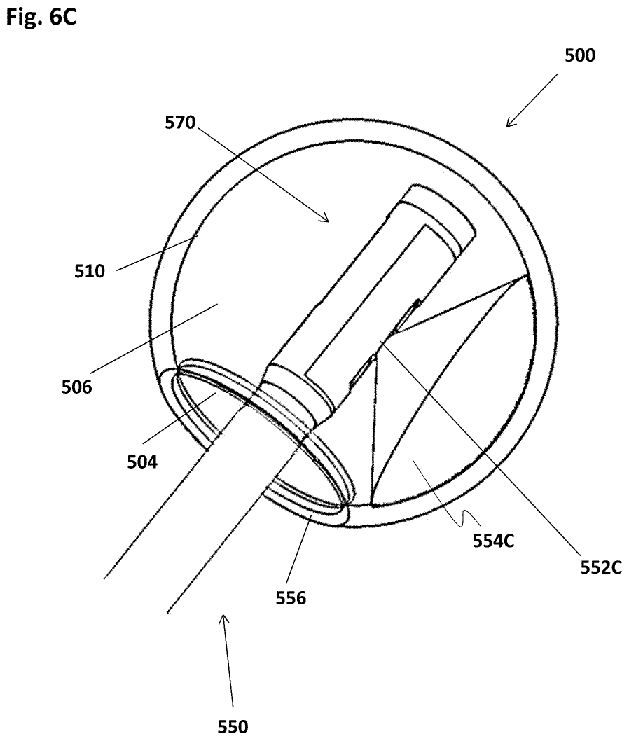

Reference is now made to FIG. 6A, which illustrates a cross section of a perspective view of a white balance apparatus of FIGS. 5A-5D having a distal tip of an endoscope which includes a plurality of cameras inserted thereto and each camera provided a field-of-view as shown in FIG. 3, according to some embodiments. As shown in FIG. 6A, a white balance apparatus 500, such as the one shown in FIGS. 5A-D includes an external shell 502, enclosing an internal space 506. Via an opening 504, a distal tip 570 of an endoscope 550 is inserted, such that the distal tip 570 of the endoscope 550 is located within the internal space 506. Endoscope distal tip 570 is similar, for example, to the one illustrated in FIG. 3. Once the distal tip 570 is correctly positioned within white balance apparatus 500, the white balance process may be executed. In order to facilitate the correct positioning of the distal tip 570 within the internal space 506 of the white balance apparatus 500, a suitable apparatus indicator 508 is located on the opening 504 of white balance apparatus 500. The apparatus indicator 508 may be any one or more types of indicators, including, for example, a visual indicator (for example, marking, such as, lines, numbers, symbols, and the like), a physical indicator (for example, a groove, a ridge, a rim, a stopper, and the like), and/or a sensor (such as, a visual sensor, proximity sensor, and the like). The apparatus indicator 508 is configured to mark and in some instances even dictate the correct positioning of the distal tip 570 within the white balance apparatus 500. In some embodiments, a corresponding shaft indicator, such as a shaft indicator 556, may be placed on the shaft of the endoscope, to match or align or otherwise communicate with the corresponding apparatus indicator 508 of the opening 504 of white balance apparatus 500. For example, the shaft indicator 556 may include a line marking, such that when the shaft indicator marking and the corresponding apparatus indicator 508 are aligned, it is indicative that the distal tip 570 is correctly positioned. For example, the shaft indicator 556 may associate with the apparatus indictor 508 by physical means, such as a groove and a matching hinge, such that when the distal tip 570 is correctly positioned, the two indicators align and match and may physically associate to indicate correct positioning. For example, the shaft indicator 556 may be an indicator which is recognizable by the corresponding sensor indicator on the white balance apparatus. For example, the sensor may be an electronic proximity sensor. For example, the sensor may be a magnetic or electromagnetic sensor, such that a magnet or electromagnet on the opening may interact with a metal strip (indicator) on the endoscope 550. For example, the sensor may be an optical sensor and the shaft indicator may include a corresponding marking identifiable by the optical sensor, including, for example, a reflective surface, a fluorescent surface, a colored surface, and the like. For example, the sensor may be an acoustic sensor. According to some embodiments, as illustrated in FIG. 6A, the correct positioning of the distal tip 570 within the white balance apparatus 500 is dictated by a distance of each of the plurality of cameras on the distal 570 tip of the endoscope 550 from an internal shell surface 510. In some embodiments, the distance is determined based on the working distance of each of the plurality of cameras. Thus, according to some exemplary embodiments, the distance between an outer window/cover/lens of a camera and the internal shell surface of the white balance apparatus is in the range of about 1-150 millimeters, or any subranges thereof. In some embodiments, the distance is in the range of about 15-125 millimeters. In some embodiments, the distance is in the range of about 1-100 millimeters. In some embodiments, the distance is in the range of about 2-50 millimeters. In some embodiments, the distance is in the range of about 1-30 millimeters.

In some embodiments, the distance between each of the cameras and the internal shell surface is equal. In some embodiments, the distance between the cameras and the internal shell is not equal. Illustrated in FIG. 6A is distance Al, which relates to the distance between a front camera 552A and the internal shell surface 510 of the white balance apparatus 500; distance A2, which relates to the distance between side camera 552C and the internal shell surface 510 of the white balance apparatus 500, and distance A3, which indicate the distance between a second side camera (not shown) and the internal shell surface 510 of the white balance apparatus 500. In some embodiments, the distances A1, A2 and A3 are equal. In some embodiments, the distances A1, A2 and A3 are not equal.

According to some embodiments, front camera 552A provides a field of view 554A, side camera 552C provides a field of view 554C and first side camera (not shown) provides a field of view 554B. Field of views 554A, 554B and 554C correlate respectively to front HFOV 310a, first side-HFOV 310b, and second side-HFOV 310c of FIG. 3.

Reference is now made to FIG. 6B, which illustrates a cross section of a side view of a white balance apparatus of FIGS. 5A-5D having a distal tip of an endoscope which includes a plurality of cameras inserted thereto and each camera provided a field-of-view as shown in FIG. 3, according to some embodiments. As shown in FIG. 6B, into white balance apparatus 500, a distal tip 570 of an endoscope (not numbered) is inserted. The distal tip 570 includes a front camera 572A, a first side camera 572B and a second side camera 572C. Each of the cameras has its own field of view, for performing a simultaneous white balance, based on images of the internal shell surface 580, obtained by each of the cameras. The correct position of distal tip 570 within the white balance apparatus 500 facilitates the simultaneous and accurate white balance processing, by positioning the distal tip 570 such that a predetermined distance for each of the cameras from the internal shell surface is kept. As shown in FIG. 6B, the center of the cameras relative to the distal tip of the endoscope may be different.

Reference is now made to FIG. 6C, which illustrates an exemplary perspective view of a field of view of a side camera of a distal tip of endoscope of FIG. 3, which includes a plurality of cameras, inserted into a white balance apparatus of FIGS. 5A-5D, according to some embodiments. As shown in FIG. 6C, white balance apparatus 500, includes an internal space 506, in which, via opening 504, a distal tip 570 of an endoscope 550 is inserted, such that the distal tip 570 is located within the internal space 506. Shown is a side field of view 554C of side camera 552C, as it faces an internal shell surface 510 of the white balance apparatus 500. Image obtained at side field of view 554C by side camera 552C (i.e., when placed and correctly positioned within white balance apparatus 500) are used for white balance processing, by a suitable processor (as detailed herein).

According to some embodiments, the white balance apparatus as disclosed in FIGS. 5A-D may be comprised as a single element or as two elements which are associated to form a whole apparatus. In some embodiments, the white balance apparatus may be substantially spherical.

In some embodiments, the white balance apparatus may have a semi-flexible or flexible structure.

In some embodiments, the white balance apparatus may be constructed of various suitable materials, such as, for example, thermoplastic elastomer (TPE) and/or a thermoset elastomer. In some embodiments, the apparatus may be constructed of silicone, or may contain at least some silicone.

In some exemplary embodiments, the silicone may be 40 shore silicon, or the like.

In some embodiments, the white balance apparatus may be constructed by press molding, using a suitable mold configured to form an outer shell with an opening, enclosing an internal space. In some embodiments, the white balance apparatus may be constructed by injection molding. In some embodiments, the white balance apparatus may be constructed using machine processing (machining). In some embodiments, the white balance apparatus may be manufactured by 3D-printing.

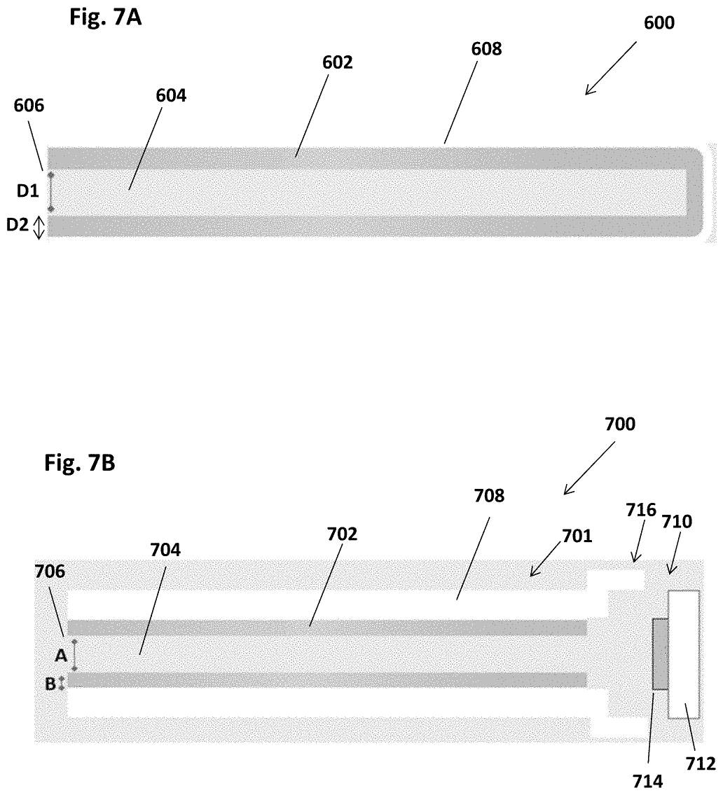

Reference is now made to FIG. 7A, which illustrates a white balance apparatus, according to some embodiments. As shown in FIG. 7A, white balance apparatus 600 includes an outer shell 602, enclosing an internal space 604. The white balance apparatus 600 includes one opening 606 through which, an endoscope tip having a plurality of cameras and a shaft of a multi-camera endoscope (such as shaft distal section 112 and elongated shaft 102 of endoscope 100 as described in FIG. 1) may be inserted into the white balance apparatus 600. As shown in FIG. 7A, the white balance apparatus 600 is cylindrical or tubular and is formed and sized to fit the endoscope tip within the internal space 604 thereof. According to some embodiments, the outer shell (wall) 602 of the white balance apparatus 600 may be rigid or semi-rigid, and may be made of glass, plastic (such as transparent plastic) and the like. An inner diameter of the white balance apparatus 600 (D1) is determined based on the diameter of the endoscope tip. In some embodiments, a thickness (D2) of the outer shell 602 is determined by the illumination light focus distance and/or the distance that the light may be diffused uniformly across each of the camera's field of view (not shown). In order to allow white balance processing, the white balance apparatus 60 may include an external cover or coating 608. The external coating 608 may include any type of suitable coating. For example, the coating may be white coating. The coating may include paint, paper, plastic, silicon, Polytetrafluoroethylene (PTFE), and the like. In some embodiments, the cover may be in the form of a sleeve. In some exemplary embodiments, the coating may include Teflon coating. In some embodiments, the white balance apparatus 600 may be disposable. In some embodiments, the white balance apparatus 600 may be reusable. In some embodiments, the white balance apparatus 600 may be sterilizable, in particular, if the outer shell 602 is made of sterilizable material, such as, glass. Further, in some embodiments, the opening 606 may include an indicator to facilitate correct positioning of the endoscope tip within the internal space 604 of the white balance apparatus 600. In some embodiments, the indicator may include visual indicator, sensor, tactile indicator, and the like. In some embodiments, the white balance apparatus 600 is constructed to ensure maximal fit with the endoscope tip to ensure the performing of a white balance process for images obtained simultaneously from the plurality of cameras located at the endoscope tip.

Reference is now made to FIG. 7B, which illustrates a white balance apparatus, according to some embodiments. As shown in FIG. 7B, white balance apparatus 700 comprises a white body apparatus body 701 and a removal cap (cover) 710. White body apparatus body 701 includes an outer shell 702 enclosing an internal space 704, and an external cover 708. The white balance apparatus 700 includes an opening 706 through which, an endoscope tip having a plurality of cameras and a shaft of a multi-camera endoscope (such as shaft distal section 112 and elongated shaft 102 of endoscope 100 as describes in FIG. 1) may be inserted into the white balance apparatus 700. As shown in FIG. 7B, the white balance apparatus 700 is cylindrical or tubular and is formed and sized to fit the endoscope tip within the internal space 704 thereof. As shown in FIG. 7B, the removal cap (cover) 710 may be plugged/snapped/screwed in on the opposing longitudinal side of opening 706. The removal cap 710 may be constructed of several parts/materials. For example, the removal cap 710 may be made of a first portion inner layer 714 made from material similar or identical in composition and thickness to the outer shell 702 and a second part an outer layer 712, made from material similar or identical in composition and thickness to the external cover 708. The removal cap 710 may fit into the white balance apparatus body 701 by engaging a suitable connection interface 716 within white balance apparatus body 701. The connection interface 716 may include any type of interface, including, for example, screw, fit-lock, snap, and the like. According to some exemplary embodiments, external cover 708 of white balance apparatus body 701 and outer layer 712 of removal cap (cover) 710 may be made of white PTFE and both with the same thickness. According to some exemplary embodiments, outer shell 702 of white balance apparatus body 701 and inner layer 714 of removal cap (cover) 710 may be made of glass or plastic both with the same thickness. According to some embodiments, the outer shell (wall) 702 of the white balance apparatus body 701 may be rigid or semi-rigid, and may be made of glass, plastic (such as transparent plastic) and the like. An inner diameter of the white balance apparatus (A) is determined based on the diameter of the endoscope tip. In some embodiments, a thickness B of the outer shell 702 is determined by the illumination light focus distance and/or the distance that the light may be diffused uniformly across each of the camera's field of view (not shown). In order to allow white balance processing, the white balance apparatus 700 may include external cover or coating 708. The external coating 708 may include any type of suitable coating. For example, the coating may be white coating. The coating may include paint, paper, plastic, silicon, Polytetrafluoroethylene (PTFE), and the like. In some embodiments, the apparatus may be disposable. In some embodiments, the apparatus may be reusable. In some embodiments, the apparatus may be sterilizable, in particular, if the shell is made of sterilizable material, such as, glass. In some embodiments, the apparatus may be autoclavable, in particular, made of autoclavable material, such as, glass, PTFE. Further, in some embodiments, the opening may include an indicator to facilitate correct positioning of the endoscope tip within the internal space of the white balance apparatus 700. In some embodiments, the indicator may include visual indicator, sensor, tactile indicator, and the like. In some embodiments, the white balance apparatus 700 is constructed to ensure maximal fit with the endoscope tip to ensure the performing of a white balance process for images obtained simultaneously from the plurality of cameras located at the endoscope tip. According to some embodiments, such design of the apparatus is advantageous as it allows readily customizable apparatus, allowing the body (tube) length of the apparatus to be adapted/fitted to the endoscope shaft length hence acting as an endoscope cover/protector.

According to some embodiments, the correct positioning of the endoscope tip within the white balance apparatus is dictated by a distance of each of the plurality of cameras on the endoscope tip from an internal surface of the apparatus. In some embodiments, the distance is determined based on the working distance of each of the plurality of cameras. Thus, according to some exemplary embodiments, the distance between an outer window/cover/lens of a camera and the internal space of the white balance apparatus is in the range of about 1-150 millimeters, or any subranges thereof. In some embodiments, the distance is in the range of about 15-125 millimeters. In some embodiments, the distance is in the range of about 1-100 millimeters. In some embodiments, the distance is in the range of about 2-50 millimeters. In some embodiments, the distance is in the range of about 1-30 millimeters. In some embodiments, the distance between each of the cameras and the internal space/surface is equal. In some embodiments, the distance between the cameras and the internal surface/space is not equal.

According to some embodiments, the apparatus is disposable. In some embodiments, the apparatus is reusable. In some embodiments, the apparatus is sterilizable. In some embodiments, the apparatus is autoclavable.

In some embodiments, the white balance processing is performed by a processing unit, for example, a processing unit located in a main control system (either as part of a main processing unit, as a circuit of the processing unit, or as a dedicated processing unit).

In some embodiments, the white balance processing includes obtaining images/video streams from a plurality of cameras within a distal tip of an endoscope, while being correctly placed/situated within the white balance apparatus, which is used as a reference color, typically white. The obtained reference images are then transferred/conveyed to the proceeding unit for further processing. The white balance processing of the obtained reference images is carried out, for example, by assigning values, parameters or any suitable coefficients to the obtained images, such that when color images are to be provided (for example, when the endoscope is inserted into a body cavity), suitable adjustment of intensity, color temperature, Hue, and the like, may be performed, to adjust one or more or each of the colors (depending on the optical sensors used (for example, RGB: red (R), green (G) and blue (B); CYMK (Cyan, Yellow, Magenta, Black); and/or YCMG: yellow (Ye), cyan (Cy), magenta (Mg), and green (G)) and apply the adjustment to the images/video signals generated from the cameras. In some embodiments, the white balance process (i.e., obtaining images from the cameras at the distal tip of the endoscope while being correctly positioned within the white balance apparatus) may be performed under different lighting conditions (for example, as dictated by the intensity of light generated by the illumination components). In some embodiments, the white balance process (may be performed for a desired length of time, that may be predetermined, or determined according to the progress of the processing. The time length may be, for example, in the range of about 0.2-20 seconds, or any subranges thereof.

According to some embodiments, there is provided a method for performing white balance of an endoscope having a plurality of cameras. In some embodiments, the method is for performing white balance for images obtained simultaneously from least two cameras located at a distal tip of an endoscope, the method includes: inserting the distal tip into the white balance apparatus as disclosed herein; verifying the positioning of the distal tip within the internal space of the apparatus, such that each of the cameras is placed at a predetermined distance from the internal shell surface of the internal space of the white balance apparatus; obtaining one or more images from the cameras, and processing the one or more images to perform white balance using a processor on a main control unit.

According to some embodiments, the method may include further data manipulation, calculation or processing. In some embodiments, the method may further include determining the white balance parameters or values (for each of the cameras and/or for a combination of cameras) and optionally storing at least some of the parameters in memory. According to some embodiments, the white balance process may be performed for a suitable period of time, such as, in the range of about 1-10 seconds, 2-5 seconds, or the like.

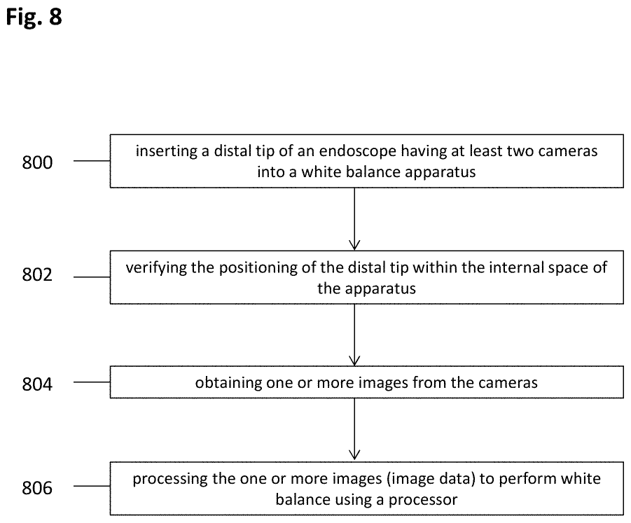

Reference is now made to FIG. 8, which illustrates steps in a method for performing white balance of images obtained from a plurality of cameras placed within a distal tip of an endoscope, according to some embodiments. As shown in FIG. 8, at step 800, the distal tip of an endoscope is inserted tip into the white balance apparatus, such that it is positioned correctly within the apparatus. Next, at step 802, the positioning of the distal tip within the internal space of the apparatus is verified. In some embodiments, the verification is such that each of the at least two cameras are placed at a predetermined distance from an internal shell surface of the space of the white balance apparatus. The placing and/or verification may be facilitated by the use of an indicator on an opening of the white balance apparatus and optionally, based on its interaction with a corresponding indicator on a shaft of the endoscope. At step 804, one or more images are obtained by the cameras. The images obtained are of the internal shell of the apparatus. At step 806, the images (image data) which has been transferred to a main control unit is being processed, to perform white balance. The processing may include any suitable white balance processing algorithms. The white balance data may be stored in memory for future reference. The main control unit which received the image data is for example main control unit 210 of medical imaging system 200 as described with reference to FIG. 2.

According to some embodiments, the method may further include presenting or displaying the image(s) obtained by the cameras during the white balance process and/or information/data/parameter related thereto.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus. In some embodiments, a computer may include of the apparatuses may include FPGA, microcontrollers, DSP and video ICS.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. In such embodiments, for example, the statement “the length of the element is equal to about 1 millimeter” is equivalent to the statement “the length of the element is between 0.99 millimeters and 1.01 millimeters”.

As used herein, according to some embodiments, the terms “substantially” and “about” may be interchangeable.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1-44. (canceled)

45. A white balance apparatus for a distal tip of an endoscope, said distal tip comprises at least two cameras, the white balance apparatus comprising an external shell at least partially enclosing an internal space, the external shell comprising an opening configured to allow the insertion of the distal tip into said internal space, such that each of said at least two cameras faces a portion of an internal shell surface of the internal space at a predefined distance therefrom, to thereby allow simultaneous white balancing of said at least two cameras.

46. The apparatus according to claim 45, wherein the opening further comprises an indicator configured to indicate the extent of insertion of the distal tip into the internal space of the apparatus.

47. The apparatus according to claim 46, wherein the indicator comprises a marking, a rim, a groove and/or a sensor.

48. The apparatus according to claim 46, wherein the indicator is configured to facilitate the positioning of the distal tip within the internal space, such that each of said at least two cameras is placed at the predetermined distance from the internal shell surface of the apparatus.

49. The apparatus according to claim 45, wherein the apparatus is substantially spherical and comprises a semi-flexible structure.

50. The apparatus according to claim 45, wherein the apparatus is made of silicone.

51. The apparatus according to claim 45, wherein the apparatus is constructed by press molding, injection molding and/or machining.

52. The apparatus according to claim 45, wherein the apparatus is substantially cylindrical or tube-like and comprises at least partially rigid walls.

53. The apparatus according to claim 52, further comprising an outer shell comprising glass and/or transparent plastic, and an external coating comprising Polytetrafluoroethylene (PTFE)

54. The apparatus according to claim 53, wherein thickness of the shell is determined based on light focus distance.

55. The apparatus according to claim 45, wherein the predetermined distance between each of the at least two cameras and the internal shell surface of the apparatus is a working distance of each of the at least two cameras.

56. The apparatus according to claim 45, wherein a distance between an outer window of each of the at least two cameras and the internal shell surface of the apparatus is in the range of about 1-150 mm.

57. The apparatus according to claim 45, wherein the at least two cameras comprise a front camera and a first side-camera, wherein the at least two cameras further comprise a second side-camera, wherein the first side-camera and the second side-camera are positioned on opposite sides of the endoscope tip, and wherein the first side-camera is positioned distally relative to the second side-camera.

58. The apparatus according to claim 45, wherein the at least two cameras provide at least about 270 degrees horizontal field-of-view (FOV) of a target area within an anatomical cavity into which the endoscope is inserted, after a white balance calibration is performed utilizing the white balance apparatus.

59. The apparatus according to claim 45, wherein each of the at least two cameras comprise a sensor configured to be associated with a main control unit comprising a white balance circuit for executing a white balance processing of images obtained by the at least two cameras, while the distal tip of the endoscope is placed within the white balance apparatus.

60. A system for white balance of at least two cameras located at a distal tip of an endoscope, the system comprising:
a white balance apparatus comprising an external shell at least partially enclosing an internal space, the external shell comprises an opening configured to allow the insertion of the distal tip into said internal space, such that each of said at least two cameras faces a portion of an internal shell surface of the internal space at a predefined distance therefrom; and
a main control unit configured to receive image data from each of the at least two cameras while placed within the apparatus, and execute a white balance processing on said images.

61. The system according to claim 60, further comprising a display configured to display one or more images and/or parameters related to the white balance processing.

62. The system according to claim 60, wherein the indicator is configured to facilitate the positioning of the distal tip within the internal space, such that each of said at least two cameras is placed at the predetermined distance from the internal shell surface of the apparatus.

63. The system according to claim 60, wherein the predetermined distance between each of the at least two cameras and the internal shell surface is a working distance of each of the least two cameras.

64. A method for performing white balance for images obtained simultaneously from at least two cameras located at a distal tip of an endoscope, the method comprising:
inserting the distal tip into a white balance apparatus comprising an external shell at least partially enclosing an internal space, the external shell comprising an opening configured to allow the insertion of the distal tip into said internal space, such that each of said at least two cameras faces a portion of an internal shell surface of the internal space at a predefined distance therefrom,
verifying the positioning of the distal tip within the internal space of the apparatus, such that each of the at least two cameras is placed at a predetermined distance from the internal shell surface of the apparatus;
obtaining one or more images from the at least two cameras, and processing the one or more images to perform white balance using a processor on a main control unit.
